(19) United States
(12) Patent Application Publication
(10) Pub. No.: US 2024/0199793 A1
(43) Pub. Date: Jun. 20, 2024

(54) THERMOPLASTIC BIODEGRADABLE ELASTOMERS AND METHODS OF USE

(71) Applicant: REGENTS OF THE UNIVERSITY OF MINNESOTA, Minneapolis, MN (US)

(72) Inventors: Wei Shen, Minneapolis, MN (US); Ronald A. Siegel, Minneapolis, MN (US); Sudipta Panja, Omaha, NE (US)

(21) Appl. No.: 18/017,948

(22) PCT Filed: Aug. 5, 2021
(86) PCT No.: PCT/US2021/044769

§ 371 Date: Jan. 25, 2023

Related U.S. Application Data
(60) Provisional application No. 63/061,830, filed on Aug. 6, 2020.

(51) Int. Cl. C08G 63/08; A61K 38/47; A61L 27/18; A61L 27/54; A61L 27/58; C08L 87/00

(52) U.S. Cl. CPC C08G 63/08; A61K 38/47; A61L 27/18; A61L 27/54; A61L 27/58; C08L 87/005; C12Y 302/01017; C08L 2201/06

(57) ABSTRACT

Methods including providing a block copolymer, the block copolymer comprising at least a polycaprolactone (PCL) block; and a second block being amorphous and having a glass transition temperature less than 30° C.; and subjecting the block copolymer to thermal processing at a temperature less than 100° C. Articles and thermoprocessing methods utilizing such block copolymers are also disclosed herein.

### CROSS-REFERENCE TO RELATED APPLICATIONS

This application claims the benefit of priority under 35 U.S.C. § 119(e) of U.S. Provisional Application No. 63/061,830, filed Aug. 6, 2020, which is incorporated by reference in its entirety.

### FIELD

This disclosure relates to, among other things, block copolymers that can be utilized in thermal processing at temperatures less than 100° C.

### BACKGROUND

Fabrication of thermoplastic, biodegradable elastomers into devices (including medical devices encapsulating bioactive agents) via thermoprocessing methods (including for example extrusion and molding) or 3D printing at temperatures lower than 100° C. (e.g. 50-60° C.) has never been reported. Thermoplastic, biodegradable elastomers that would enable such processing without losing bioactivity of bioactive agents may be quite advantageous for biomedical applications such as medical devices, for example.

### SUMMARY

In some embodiments described herein, the methods including providing a block copolymer, the block copolymer comprising at least a polycaprolactone (PCL) block; and a second block being amorphous and having a glass transition temperature less than 30° C.; and subjecting the block copolymer to thermal processing at a temperature less than 100° C.

In some embodiments described herein, the elastomeric polymer PMVL, as an example, is combined with hard polymers to produce block copolymers having soft (PMVL) and hard blocks. For example, hard block-PMVL-hard block triblock polymers are described herein. In some embodiments, the hard blocks are polycaprolactone (PCL) blocks.

One or more embodiments of the compounds, polymers, compositions or methods described herein may provide one or more advantages relative to existing compounds, polymers, compositions or methods. Additional features and advantages of the subject matter of the present disclosure will be set forth in the detailed description which follows, and in part will be readily apparent to those skilled in the art from that description or recognized by practicing the subject matter of the present disclosure as described herein, including the detailed description which follows, the claims, as well as the appended drawings.

It is to be understood that both the foregoing general description and the following detailed description present embodiments of the subject matter of the present disclosure, and are intended to provide an overview or framework for understanding the nature and character of the subject matter of the present disclosure as it is claimed. The accompanying drawings are included to provide a further understanding of the subject matter of the present disclosure, and are incorporated into and constitute a part of this specification. The drawings illustrate various embodiments of the subject matter of the present disclosure and together with the description serve to explain the principles and operations of the subject matter of the present disclosure. Additionally, the drawings and descriptions are meant to be merely illustrative, and are not intended to limit the scope of the claims in any manner.

### BRIEF DESCRIPTION OF THE DRAWINGS

FIG. 1 shows ¹H NMR spectrum of βMδVL in CDCl₃

FIG. 2 shows ¹³C NMR spectrum of βMδVL in CDCl₃

FIGS. 3A and 3B show ¹H NMR spectra of PβMδVL (FIG. 3B) and PCL₃₀-PβMδVL-PCL₃₀ (FIG. 3B) dissolved in CDCl₃ along with assigned peaks.

FIGS. 4A, 4B and 4C shows ¹³C NMR spectra of (FIG. 4A) PβMδVL, (FIG. 4B) PCL₃₀-PβMδVL-PCL₃₀, and (FIG. 4C) PCL₄₅ in CDCl₃

FIG. 5 show DSC thermograms reveal that PβMδVL is amorphous and has a glass transition temperature of −51.75° C.; the PCL endblocks are semi-crystalline and have a melting temperature below 56° C.

FIG. 6 shows ¹H NMR of spectra of PCL₁₅-PβMδVL-PCL₁₅ in CDCl₃.

FIG. 7 shows Gel permeation chromatography analysis for the synthesized polymers. The peaks of PCL₁₅-PβMδVL-PCL₁₅ and PCL₃₀-PβMδVL-PCL₃₀ shift toward smaller retention time as compared with that of PβMδVL, suggesting successful synthesis of the triblock polymers.

FIGS. 8A, 8B, 8C and 8D shows the result of uniaxial extension tests and cyclic tensile tests for the synthesized polymers. (FIG. 8A) Representative uniaxial stress-strain curves. (FIG. 8B) Stress-strain curves of PCL₁₅-PβMδVL-PCL₁₅ during cyclic loading to 50% strain. (FIG. 8C) The hysteresis energy loss. (FIG. 8D) The tensile sets during cyclic loading. All the tests were performed with a strain rate of 10 mm/min.

FIGS. 9A, 9B, and 9C shows that PCL-PβMδVL-PCL triblock elastomers are not cytotoxic. (FIG. 9A) The AlamarBlue assay of 3T3 fibroblasts seeded at 15,000 cells/cm² on the triblock elastomers and cultured for 24 h. (FIG. 9B) The AlamarBlue assay of 3T3 fibroblasts seeded at 3,000 cells/cm² on PCL₁₅-PβMδVL-PCL₁₅ and cultured for up to 5 days. (FIG. 9C) Live-dead staining for 3T3 fibroblasts seeded on PCL₁₅-PβMδVL-PCL₁₅ and cultured for 1 and 5 days. Statistical analysis was performed using the One Way ANOVA test. *P<0.05 and **P<0.01 compared with the 1-day data in (FIG. 9B).

FIG. 10 shows thermoplastic PCL₁₅-PβMδVL-PCL₁₅ was 3D-printed into two structures through fused deposition modeling (FDM) at 60° C.

FIGS. 11A, 11B, and 11C shows bright-field images of (FIG. 11A) PCL₁₅-PβMδVL-PCL₁₅, (FIG. 11B) PCL₁₅-PβMδVL-PCL₁₅ containing lysozyme, which was encapsulated through melt-blending at 60° C., and (FIG. 11C) PCL₁₅-PβMδVL-PCL₁₅ containing lysozyme, which was encapsulated through solvent casting.

FIG. 12 shows uniaxial stress-strain behavior of pristine PCL₁₅-PβMδVL-PCL₁₅ and PCL₁₅-PβMδVL-PCL₁₅ containing 18.25 wt % lysozyme, which was encapsulated through melt-blending at 60° C.

FIG. 13 shows the activity of the lysozyme encapsulated in PCL₁₅-PβMδVL-PCL₁₅ through double heat processing at 60° C. (melt-blending and hot-pressing) compared with that of pristine lysozyme and DCM-treated lysozyme. (statistical analysis shows no significant difference among three groups).

FIG. 14 shows the weight loss profiles of PCL₃₀-PβMδVL-PCL₃₀ in PBS and a lipase solution at 37° C. over 8 weeks.

### DETAILED DESCRIPTION

This disclosure relates to, among other things, polymers such as block copolymers that include a block containing polycaprolactone (PCL) and a second block that is amorphous and having a low glass transition temperature, such as less than 30° C. The copolymers may be useful in thermal processing methods that occur at temperatures less than 100° C. The copolymers generally can be described as biodegradable, elastomeric and processable at generally lower temperatures.

Block copolymers allow for the properties of different polymer materials to be extended by combining two or more different polymers in a final product. The properties of a block copolymer can be tailored by tuning the length and the type of blocks used. In some embodiments, disclosed block copolymers may be biodegradable, elastomeric, and melt at a temperature below 100° C., for example.

In various embodiments, block copolymers disclosed herein can be a di-block copolymer (A-B), a tri-block copolymer (e.g., A-B-C, A-B-A or B-A-B), or a multiblock copolymer [e.g., (A-B)ₙ].

In various specific illustrative embodiments, the block copolymers described herein are thermoplastic elastomers that combine a poly (beta-methyl-delta-valerolacone) (PMVL) elastomer with a hard block thermoplastic polymer. Such thermoplastic elastomers behave like elastomers at temperatures between the glass transition temperatures of PMVL and the melting temperature of the hard block and can be processed like thermoplastics at temperatures below the melting temperature of the hard block.

In some embodiments, one or more hard blocks may be combined with one or more soft blocks to form disclosed block copolymers. In some embodiments, one of the blocks may be polymerized first via any useful method, including for example ring-opening polymer (ROP) and then at least a second block may be added via any useful method, including for example ROP. Other useful methods of polymerization may be utilized for the polymerization of either of the blocks.

In some embodiments disclosed block copolymers comprise a PCL block and a second block that is amorphous and has a glass transition temperature less than 30° C., such as a PMVL block.

Some disclosed copolymers include polycaprolactone (PCL) blocks. PCL melts at a temperature between 50° C.-60° C. PCL is crystalline or semicrystalline at room temperature (e.g., at about 23° C.), providing physical junctions that allow reversibility. The percent crystallinity and molecular weight of the PCL blocks dictate the melting temperature of the particular PCL blocks utilized. This melting temperature is lower, in some cases significantly lower, than the melting temperature of other polyesters, for example polylactic acid (PLA).

The PCL blocks can have any suitable length. In some embodiments, a PCL block (e.g., an individual end block) has a mass average molar mass (M_{w}) of from about 0.5 to about 100 kDa, such as from about 1 to about 50 kDa or from about 2 to about 30 kDa. In general, a block will have a Mw of about 0.25 kDa or greater.

Polymerization of caprolactone may occur in solution, in the melt, or as a suspension.

In disclosed block copolymers, the melting temperature of the PCL block(s) may determine the melting temperature of the overall block copolymer. Such a relatively low melting temperature may offer advantages, in some embodiments several advantages: it allows the polymers to be thermo-processed at temperatures lower than 100° C., in some cases much lower than 100° C. (e.g., 50° C.-60° C.); it allows molecular agents to be mixed with melted polymers at temperatures much lower than 100° C., in some cases much lower than 100° C. (e.g., 50° C.-60° C.). The ability to combine molecular agents with the block copolymers at such low temperatures affords avenues for encapsulating the molecular agents, for example bioactive molecules, under conditions that don't threaten the bioactivity thereof.

Disclosed block copolymers can also include at least a second block that is amorphous and has a low glass transition temperature, such as a glass transition temperature less than 30° C. In some disclosed block copolymers, the second block can influence the elastic nature of the block copolymer and can contribute to low temperature properties and extensibility of the block copolymer. In some embodiments, the second block can be a PMVL block. Illustrative second blocks can also include, for example amorphous poly(caprolactone-co-lactide), poly(lactide-co-trimethylene carbonate), polyurethane, poly(trimethylene carbonate), poly(ester ether), polyhydroxyalkanoates, and random copolymers formed from caprolactone and any other monomers that disrupt the crystalline structure of the resulting polymers. In some embodiments, the second block can also include any other monomers that can function to disrupt the crystalline structure of the resulting polymers. In some embodiments, the PMVL block has a Tg of less than −30° C. For example, the PMVL block can have a Tg of less than −40° C. or less than −50° C. In many embodiments, the PMVL block has a Tg greater than −100° C. In some embodiments, the PMVL block has a Tg of about −51° C.

PMVL blocks may be formed in any suitable manner. For example, PMVL may be formed via ring-opening transesterification polymerization (ROTEP) of MVL employing an appropriate initiator and catalyst. Any suitable catalyst may be employed. Examples of suitable catalysts include metal catalysts or organocatalysts, such as tin octoate; triethyl aluminum; zinc dibutoxide; titanium tetrabutoxide; triazobicyclodecene (TBD); 1,4-Benzene dimethanol (BDM); diphenyl phosphate (DPP); and the like.

Any suitable initiator may be employed. For example, the initiator may be an organometal (e.g. alkyl lithium, alkyl magnesium bromide, alkyl aluminum, etc.), a metal amide, an alkoxide, a phosphine, an amine, an alcohol, or the like. In some embodiments, the initiator is an alcohol. The initiator may be monofunctional or multi-functional. Examples of suitable ring opening polymerization initiators include benzyl alcohol; 1,4 benzene dimethanol; and the like. One of skill in the art will understand that the ratio of monomer to initiator may be varied to obtain polymers of different molecular weights.

Polymerization of MVL may occur in solution, in the melt, or as a suspension.

The PMVL blocks can be of any suitable length. In some embodiments, a PMVL block has a mass average molar mass (M_{w}) of 0.25 kDa or greater, such as 0.5 kDa or greater, or 1 kDa or greater. In some embodiments, a PMVL block has M_{w} of from about 1 to about 500 kDa, such as from about 2 to about 250 kDa or from about 3 to about 100 kDa.

Non-PMVL blocks, such as PCL blocks, can be formed in any suitable manner, such as polymerization of one or more monomers, or the like. A variety of methods for producing such block copolymers are described herein. Such methods include polymerizing monomers from a living PMVL polymer or a telechelic PMVL block and coupling a PMVL block to a PCL block.

Disclosed copolymers may be biodegradable, elastic (e.g., stretchable) and biocompatible. An important known polymer with these characteristics is poly(glycerol sebacate (PGS). A significant disadvantage of PGS is that it is a thermosetting polymer so it must be cured. It is cured at temperatures greater than 130° C. under vacuum for two to three days. Such conditions are not amenable to use with temperature sensitive materials such as biological or bioactive agents.

It will be understood that the above are merely examples of suitable hard blocks and methods for forming hard blocks. One of skill in the art would understand that other hard blocks may be used and may be readily synthesized.

Block copolymers described herein can be formed in any suitable manner. In some embodiments, non-PMVL blocks (or PMVL blocks) are added to PMVL blocks (or non-PMVL blocks) via living polymerization. For example, monomers forming a non-PMVL block (or a PMVL block) can be added to a living PMVL (or non-PMVL), preferably at equilibrium. Preferably, the catalyst employed for the polymerization of the living polymer is suitable for use in polymerizing the later added monomers for forming the other block or blocks. Of course, one or more additional catalysts may be added as appropriate.

In some embodiments, an ABA triblock copolymer is formed via living polymerization. Monomers for forming the B block can be polymerized and monomers forming the A blocks can be added to the living B block polymer. By way of example, caprolactone can be added to a living PMVL. In some embodiments, triazobicyclodecene (TBD) can be used to catalyze the MVL polymerization and polymerization of the caprolactone

In some embodiments, non-MVL monomers (or MVL monomers) may be added to a previously-purified telechelic PMVL (or telechelic poly(non-MVL)). For example, the purified telechelic PMVL (or telechelic poly(non-MVL)) and non-MVL monomer (or MVL) can be combined in solution or in melt/bulk and a catalyst can be added to cause polymerization of the non-MVL monomer (or MVL) from one or more ends of the previously-purified telechelic PMVL (or telechelic poly(non-MVL)). The amount of added non-MVL monomer (or MVL) added can depend on the target composition of the block polymer.

In some embodiments, an ABA block copolymer can be formed by adding caprolactone and a catalyst to a telechelic PMVL in solution or in melt/bulk to cause polymerization of the caprolactone monomer from the ends of the previously-purified telechelic PMVL.

In some embodiments, non-PMVL oligomers, such as hard block oligomers, are reacted with telechelic PMVL to form a copolymer. Alternatively PMVL-(PCL) coblocks can be synthesized to form multiblocks. Coupling agents, such as those generally known in the art, can be used to couple the oligomers to form a block copolymer or to couple the coblocks to form the multiblocks. By way of example, multiblocks can be synthesized conveniently from dihydroxy telechelic PCL-PMVL-PCL triblock using a coupling approach. Coupling agents that can be used for this purpose include diacid chlorides, such as for example terephthalic acid and sebacoyl chloride, diisocyantes, such as for example methylene diisocyante, and divinyl adipate.

A block copolymer described herein may include any suitable amount of PCL blocks, any suitable amounts of PMVL and even non-PMVL blocks. For example, the weight percent of PCL blocks in a disclosed block copolymer (e.g., a PCL-PMVL-PCL ABA block copolymer) can be from about 5% to about 95%, such as from 10% to about 90%, from about 20% to about 70%, or from about 25% to about 60%.

The lengths and percentages of non-PMVL block polymer and PMVL may be modified to achieve desired properties of a block copolymer (e.g., a PLA-PMVL-PLA ABA block copolymer). Hard blocks tend to be hard, while PMVL tends to be soft. By varying the percent and length of hard blocks and PMVL the properties of the resulting copolymer can be tuned as desired. For example, incorporation of relatively small amounts of the hard block polymer, soft highly elastic polymeric material may result. By way of further example, incorporation of higher amounts of the hard block polymer, stiffer and ductile plastics may result.

In some embodiments, a block copolymer described herein may have an elastic modulus of from about 0.01 MPa to about 2500 MPa, such as from about 0.2 MPa to about 1500 MPA, from about 0.5 MPa to about 500 MPa, from about 1 MPA to about 400 MPa, or from about 1.5 MPa to about 300 MPa. In embodiments, block copolymer described herein has a percent elongation of from about 5% to about 5000%, such as from about 10% to about 4500%, from about 100% to about 4000%, from about 200% to about 3000%, or from about 300% to about 2000%.

Disclosed block copolymers may be useful in various thermal processing methods. Useful thermal processing methods are those that take place at temperatures less than 100° C., less than 70° C., less than 65° C., or less than 60° C., or in a range from 50 to 60° C., for example.

Useful thermal processing methods may include, but are not limited to three dimensional (3D) printing, extrusion, molding. Any of various thermal processing methods may be used to form an article or a device comprising a disclosed block copolymer. Disclosed block copolymers may or may not be utilized to encapsulate a material, such as a temperature sensitive material. Useful or advantageous materials may include temperature sensitive materials, such as for example, bioactive agents (e.g., proteins and small molecules that stimulate biological responses for example), drugs, etc. A specific example of a device that may advantageously be made using disclosed block copolymers include soft stents that encapsulate a bioactive agent that would lose activity when exposed to a temperature about 65° C. for example.

Useful bioactive agents can be selected from: growth factors, cytokines, small molecules and combinations thereof. Useful growth factors, cytokines, or combinations thereof are selected from: vascular endothelial growth factor (VEGF), Platelet-derived growth factor (PDGF), basic fibroblast growth factor (bFGF), acidic fibroblast growth factor (aFGF), hepatocyte growth factor (HGF), insulin-like growth factor-1 (IGF-1), stromal-cell derived factor 1 (SDF-1), nerve growth factor (NGF), neurotrophin 3 (NT-3), brain-derived neurotrophic factor (BDNF), glial-derived neurotrophic factor (GDNF), glial growth factor (GGF), ciliary neurotrophic factor (CNTF), leukemia inhibitory factor (LIF), growth differentiation factor 5 (GDF-5), Erythropoietin (EPO), interleukin-4 (IL-4), interleukin-2 (IL-2), interferon gamma (IFN-γ), and combinations thereof. Useful small molecules can be selected from the group consisting of: paclitaxel, carmustine, gemcitabine, histrelin, leuprolide, goserelin, corticosteroids, simvastatin, risperidone, tacrolimus, vancomycin, lidocaine, buprenorphine, hydromorphine, levonorgestrel, estradiol, etonogestrel, etonogestrel, and combinations thereof.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise.

As used in this specification and the appended claims, the term “or” is generally employed in its sense including “and/or” unless the content clearly dictates otherwise. The term “and/or” means one or all of the listed elements or a combination of any two or more of the listed elements.

As used herein, “have”, “having”, “include”, “including”, “comprise”, “comprising” or the like are used in their open ended sense, and generally mean “including, but not limited to”. It will be understood that “consisting essentially of”, “consisting of”, and the like are subsumed in “comprising” and the like. As used herein, “consisting essentially of,” as it relates to a composition, product, method or the like, means that the components of the composition, product, method or the like are limited to the enumerated components and any other components that do not materially affect the basic and novel characteristic(s) of the composition, product, method or the like.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure, including the claims.

Also herein, the recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” a particular value, that value is included within the range.

As used herein, the term “about” encompasses the range of experimental error that occurs in any measurement.

Any patent or non-patent literature cited herein, including provisional patent applications, is hereby incorporated herein by reference in its entirety to the extent that it does not conflict with the disclosure presented herein.

One of skill in the art will understand that one or more materials, articles, compositions, processes, and the like disclosed in one or more patent and non-patent literature cited herein can be modified to obtain a process, monomer, polymer or the like described herein.

In the description above several specific embodiments of compounds, compositions, products and methods are disclosed. It is to be understood that other embodiments are contemplated and may be made without departing from the scope or spirit of the present disclosure. The description, therefore, is not to be taken in a limiting sense.

In the following non-limiting examples that provide illustrative embodiments of the compositions, food products, methods and sweetness enhancers described above. These examples are not intended to provide ay limitation on the scope of the disclosure presented herein.

### EXAMPLES

### Example 1: PMVL and PCL-PMVL-PCL Block Copolymers

### Materials and Methods

Biosynthesis of MVL: MVL was synthesized generally as described in Example 2 of WO2014/172596A2, entitled BIOSYNTHETIC PATHWAYS AND PRODUCTS, published on 23 Oct. 2014.

Polymer Syntheses: Unless otherwise stated herein, reagents were purchased from Sigma-Aldrich (St. Louis, MO) and were used without further purification. All reagents used for polymer synthesis were stored and handled in a glovebox under a nitrogen atmosphere. Copper chromite (Cu₂Cr₂O₅), phosphorus pentoxide (P205), calcium hydride (CaH2), ε-caprolactone, poly(caprolactone) (PCL₄₅; Mn=45 kDa), 1,4-benzenedimethanol (BDE), diphenyl phosphate (DPP), tin(II) 2-ethylhexanoate (Sn(Oct)2), dichloromethane (DCM), methanol (MeOH), tetrahydrofuran (THF), anhydrous toluene, lipase (from Thermomyces lanuginosus solution, activity >=100,000 U/g), and lysozyme (activity 22,000 U/mg) were purchased from Sigma-Aldrich. 3-methyl-1,5-pentanediol was purchased from the TCI America. Fetal bovine serum (FBS), penicillin-streptomycin (10,000 U/mL), and the lysozyme activity assay kit were purchased from Thermo Fisher Scientific. Alamar Blue® was purchased from Bio-Rad. The live-dead assay reagents, ethidium homodimer III and calcein AM, were purchased from Biotium (USA).“Triazobicyclodecene (TBD) (98%, Sigma-Aldrich) was purchased and purified by sublimation. 1,4-Benzene dimethanol (BDM) (99%, Acros Organics) and diphenyl phosphate (DPP) (99%, Sigma-Aldrich) were purchased and dried under vacuum at room temperature for a minimum of 48 hours prior to use. Benzyl alcohol (99%, Sigma-Aldrich) was purchased and used without additional purification._{D,L}-Lactide was a kind gift from Ortec Incorporated and was used as received. L-Lactide was a kind gift from Natureworks and was recrystallized twice from dry toluene and dried prior to use. Toluene and dichloromethane (DCM) was passed through a home-built solvent purification system, which includes a column of activated alumina and a column of molecular sieves operated under a positive pressure of nitrogen gas. Anhydrous methanol (Sigma-Aldrich) and chloroform (Fisher) were purchased and used as received. Triethylamine (99.5%, Macron), and benzoic acid (99.5%, Fisher) used to quench DPP and TBD catalyzed reactions, respectively were also purchased and used as received. Glass and TEFLON components used for polymer synthesis were dried in an oven at 100° C. for a minimum of 6 hours immediately prior to use.

Synthesis of β-methyl-δ-valerolactone (βMδVL) and poly(b-methyl-δ-valerolactone) (P βMδVL)

The cyclic beta-methyl-delta-valerolactone (MVL) monomer was synthesized from 3-methyl-1,5-pentanediol in the presence of copper chromite via a previously reported procedure (Brutman, J. P.; De Hoe, G. X.; Schneiderman, D. K.; Le, T. N.; Hillmyer, M. A. Renewable, Degradable, and Chemically Recyclable Cross-Linked Elastomers. Industrial & Engineering Chemistry Research 2016, 55 (42), 11097-11106, DOI: 10.1021/acs.iecr.6b02931) with a slight modification. The reaction was allowed to proceed at 210° C. overnight. The resulting MVL was purified from the reaction mixture through distillation and characterized with ¹H and ¹³C NMR spectroscopy.

Synthesis of poly(β-methyl-δ-valerolactone) (P βMδVL)

Bifunctional telechelic poly(β-methyl-δ-valerolactone) (PβMδVL) was synthesized through ring-opening polymerization of MVL with 1,4-benzenedimethanol (BDE) as an initiator and diphenyl phosphate (DPP) as the catalyst (Brutman, J. P.; De Hoe, G. X.; Schneiderman, D. K.; Le, T. N.; Hillmyer, M. A. Renewable, Degradable, and Chemically Recyclable Cross-Linked Elastomers. Industrial & Engineering Chemistry Research 2016, 55 (42), 11097-11106, DOI: 10.1021/acs.iecr.6b02931; and Xiong, M.; Schneiderman, D. K.; Bates, F. S.; Hillmyer, M. A.; Zhang, K. Scalable production of mechanically tunable block polymers from sugar. Proceedings of the National Academy of Sciences of the U.S. Pat. No. 2,014,111 (23), 8357-62, DOI: 10.1073/pnas.1404596111). The polymerization was allowed to continue at room temperature with stirring for 18 h. To purify the product, the reaction mixture was dissolved in dichloromethane (DCM), and the solution was slowly added to an excess volume of cold methanol (MeOH) to allow precipitation of PβMδVL. The purified PβMδVL was dried and characterized with NMR (¹H and ¹³C) and gel permeation chromatography (GPC).

### Synthesis of Poly(Caprolactone)-PβMδVL-Poly(Caprolactone) (PCL-PβMδVL-PCL) Triblock Copolymers

Triblock PCL-PβMδVL-PCL polymers were synthesized through ring-opening polymerization of caprolactone using bifunctional PβMδVL as a macroinitiator (Panja, S.; Dey, G.; Bharti, R.; Mandal, P.; Mandal, M.; Chattopadhyay, S. Metal Ion Ornamented Ultrafast Light-Sensitive Nanogel for Potential in Vivo Cancer Therapy. Chemistry of Materials 2016, 28 (23), 8598-8610, DOI: 10.1021/acs.chemmater.6b03440; and Panja, S.; Dey, G.; Bharti, R.; Kumari, K.; Maiti, T. K.; Mandal, M.; Chattopadhyay, S. Tailor-Made Temperature-Sensitive Micelle for Targeted and On-Demand Release of Anticancer Drugs. ACS applied materials & interfaces 2016, 8 (19), 12063-74, DOI: 10.1021/acsami.6b03820). In a nitrogen atmosphere glove box, PβMδVL was dissolved in dry toluene at 130° C. After the solution was cooled to room temperature, caprolactone and the catalyst tin(II) 2-ethylhexanoate (Sn(Oct)₂) were added and the reaction was allowed to proceed at 110° C. for 4 h under stirring. To purify the product, the reaction mixture was dissolved in DCM, and the solution was slowly added to an excess volume of cold MeOH to allow precipitation of the polymer. The purified triblock polymer was dried and characterized with NMR (H and ¹³C) and GPC.

### Polymer Characterization

The chemical structures of the polymers were analyzed by using a 400 MHz Bruker Advance III NMR spectrometer. The molecular weights and dispersities (D) of the polymers were determined using GPC equipped with a Wyatt Technology DAWN DSP multi-angle light scattering (MALS) and a Wyatt Optilab EX RI detector.

Thermal properties of the polymers were characterized using differential scanning calorimetry (DSC) on a TA Discovery Series DSC instrument. Crystallinity of each polymer was calculated by comparing its melting enthalpy with the melting enthalpy of fully crystalline PCL (139.5 J/g) (Table 1) (Zachmann, U. K. a. H. G. New Aspects Concerning the Structure and Degree of Crystallinity in High-Pressure-Crystallized Poly(ethylene terephthalate). Macromolecules 1996, 29, 6019-6022; and Schneiderman, D. K.; Hill, E. M.; Martello, M. T.; Hillmyer, M. A. Poly(lactide)-block-poly(F-caprolactone-co-F-decalactone)-block-poly(lactide) copolymer elastomers. Polymer Chemistry 2015, 6 (19), 3641-3651, DOI: 10.1039/c5py00202h). To characterize the mechanical properties of the triblock polymers, the materials were processed into 0.2 mm thick films through heat pressing at 60° C. Each material was sandwiched between two TEFLON sheets and placed on a hot-press instrument (Heated Press, Carver Model-CH (4386) hydraulic laboratory press, United States) preheated at 60° C. The sample was pressed at 350 kPa and 60° C. for 5 min, and then removed from the instrument and allowed to cool down to room temperature. Dog-bone shaped specimens (14 mm long; 3.4 mm wide; 0.2 mm thick) were cut with a punching die.

Uniaxial extension tests were conducted with a strain rate of 10 mm/min on a uniaxial tensile tester instrument (Shimadzu AGS-X). To reveal the strain to failure, the tests were performed until the specimens broke. The Young's modulus (E) was calculated from the slope of the stress-strain curve at lower strain region (0-5%) after linear regression analysis (Du, Y.; Yu, M.; Ge, J.; Ma, P. X.; Chen, X.; Lei, B. Development of a Multifunctional Platform Based on Strong, Intrinsically Photoluminescent and Antimicrobial Silica-Poly(citrates)-Based Hybrid Biodegradable Elastomers for Bone Regeneration. Advanced Functional Materials 2015, 25 (31), 5016-5029, DOI: 10.1002/adfm.201501712). Tensile hysteresis and reversibility tests were performed with a strain rate of 10 mm/min and a maximal strain of 50% for 20 cycles.

Encapsulation of lysozyme in PCL-PβMδVL-PCL

Lysozyme was encapsulated in the PCL₁₅-PβMδVL-PCL₁₅ polymer through melt-blending in a twin-screw extruder (Microcompounder, DACA Instruments) (Kim, H.; Miura, Y.; Macosko, C. W. Graphene/Polyurethane Nanocomposites for Improved Gas Barrier and Electrical Conductivity. Chemistry of Materials 2010, 22 (11), 3441-3450, DOI: 10.1021/cm100477v). The polymer was first hot-pressed (350 kPa; 60° C.) to a 0.2 mm thick film as aforementioned, and the film was rolled with lysozyme powders (with a targeted polymer to lysozyme weight ratio of 5:1) and then fed into the extruder. The melt-blending was performed at 60° C. under N₂ atmosphere for 10 min, with the rotation speed of the twin-screw maintained at 100 rpm. The melt-blended sample was hot-pressed to a 0.2 mm thick film using the aforementioned condition.

The distribution of lysozyme powders in the film was examined on an Olympus IX70 Inverted System Microscope with a 10X objective (0.25 NA). To evaluate whether the multiple heat processing procedures affected lysozyme activity, 2 g of the film was dissolved in 20 mL DCM, followed by extraction of the lysozyme with 10 mL of water. The lysozyme solution was lyophilized, and the resulting powder was weighed; the lysozyme loading content was determined by calculating the ratio of this weight to 2 g. The activity of the extracted lysozyme powder was determined using a lysozyme activity assay kit according to the manufacturer's instruction (Thermo Fisher Scientific, USA). The activities of pristine lysozyme and DCM-treated lysozyme were also measured for comparison. The DCM-treated lysozyme was prepared by dispersing pristine lysozyme in DCM, followed by extraction with water and lyophilization.

### 3D-Printing

Demonstration of 3D-printing of PCL-PβMδVL-PCL polymers at 60° C. was performed on a CELLINK's BIO-X 3D-printing instrument with a thermoplastic printhead setup. The patterns to be printed were designed in SolidWorks, and the files were uploaded to the instrument. The polymer film was fed into the heating chamber of the printhead and heated at 60° C. for 10 min, and the polymer melt was printed at 60° C. with a speed of 0.5 mm/min under a pressure of 200 kPa.

### Cell Viability Assessment

To evaluate cytotoxicity of the polymers, the materials were hot-pressed into 0.2 mm thick films. Specimens with a diameter of 6 mm were punched and mounted to the bottom of 96-well culture plates with autoclaved vacuum grease. Tissue culture polystyrene was used as a control. The specimens were incubated with 500 U/mL penicillin-streptomycin solution for 2 h for sterilization, followed by washing with phosphate buffer saline (PBS) 3 times. Fibroblasts (NIH/3T3) were plated at a density of 5,000 cells/well and cultured in DMEM medium supplemented with 10% fetal bovine serum (FBS) and 100 U/mL penicillin-streptomycin for 24h, followed by the Alamar Blue assay to determine cell viability. In brief, the Alamar Blue reagent was added to each well (10% v/v) and incubated for 4h, and the fluorescence intensity of the medium (excitation at 560 nm; emission at 590 nm) was measured on a BioTek™ Cytation™ 3 Cell Imaging Multi-Mode plate reader (Moghanizadeh-Ashkezari, M.; Shokrollahi, P.; Zandi, M.; Shokrolahi, F.; Daliri, M. J.; Kanavi, M. R.; Balagholi, S. Vitamin C Loaded Poly(urethane-urea)/ZnAl-LDH Aligned Scaffolds Increase Proliferation of Corneal Keratocytes and Up-Regulate Vimentin Secretion. ACS applied materials & interfaces 2019, 11 (39), 35525-35539, DOI: 10.1021/acsami.9b07556). To evaluate cell viability at longer time, fibroblasts were seeded at a density of 1,000 cells/well, and the Alamar Blue assay was conducted at day 1, day 3 and day 5 of culture. Each experiment was conducted in triplicate.

Cytotoxicity of the polymers was further examined using live-dead cell staining. Fibroblasts were plated at a density of 1,000 cells/well and cultured for 1 or 5 days, followed by staining with ethidium homodimer and calcein AM for 30 min. Each polymer specimen was placed on a glass slide with the cell side facing down, and fluorescence images were acquired on an Olympus IX70 Inverted System fluorescence microscopy.

### Degradation Kinetics

Specimens with a diameter of 8 mm were punched from 2 mm thick polymer films, and the initial weight of each specimen was recorded. Each specimen was placed in a 15 mL Falcon tube containing 5 mL of 2000 U/mL lipase solution (Thermomyces lanuginosus) or PBS, and incubated at 37° C. The solution in each tube was replaced with fresh solution weekly. At various time points, the specimens were washed with methanol, dried, and weighed. The weight loss was calculated by subtracting the final weight from the initial weight.

### Results

Synthesis of β-methyl-δ-valerolactone (PβMδVL)

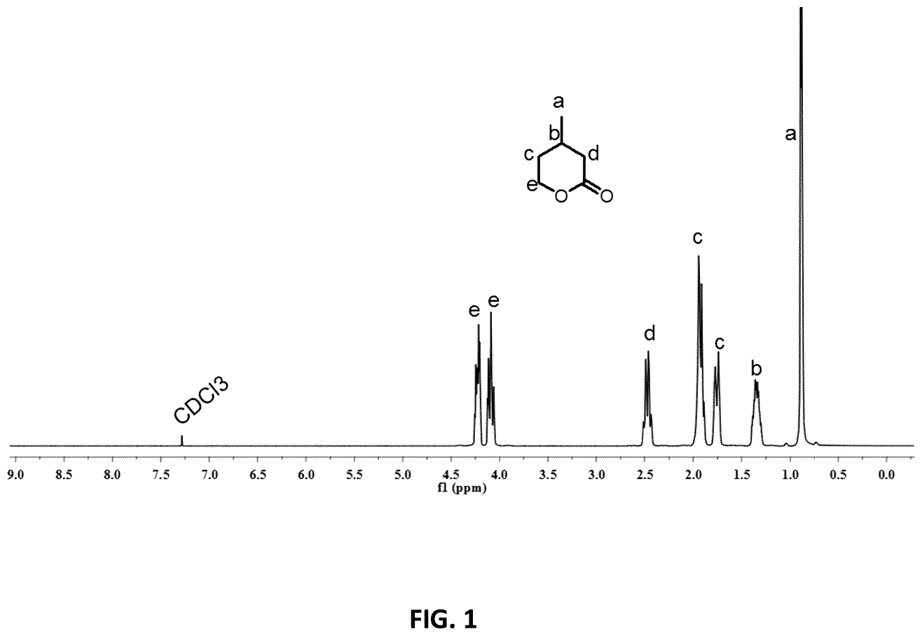

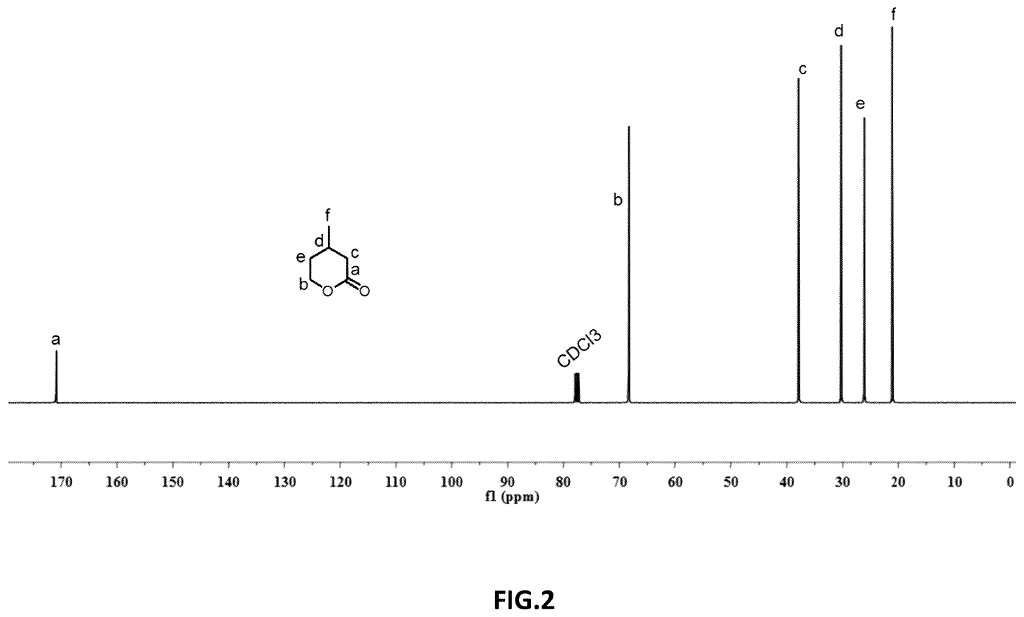

Successful synthesis of the lactone monomer β-methyl-δ-valerolactone (βMδVL) from 3-methyl-1,5-pentanediol was confirmed by ¹H and ¹³C NMR analyses. The product displayed ¹H NMR signals at 4.23 and 4.08 ppm for the two anomeric protons (—CH₂—O; e), at 2.44 ppm (—CH₂—CO; d) and 1.94 and 1.74 ppm (—CH₂—CH₂O—, c) for the methylene protons, at 1.34 ppm for the methine proton (—CH(CH₃)CH₂—; b), and at 0.84 ppm for the methyl protons (—CH₃; a) (FIG. 1). The ¹³C NMR signals at 170.86 (—COO—; a), 68.28 (—CH₂O—; b), 37.90 (—CH₂CO—; c), 30.29 (—CH₂—; d), 26.17 (—CH(CH₃)—; e), and 20.98 (—CH₃; f) were assigned to their corresponding carbons as shown in FIG. 2.

Synthesis and characterization of poly(β-methyl-δ-valerolactone) (PβMδVL)

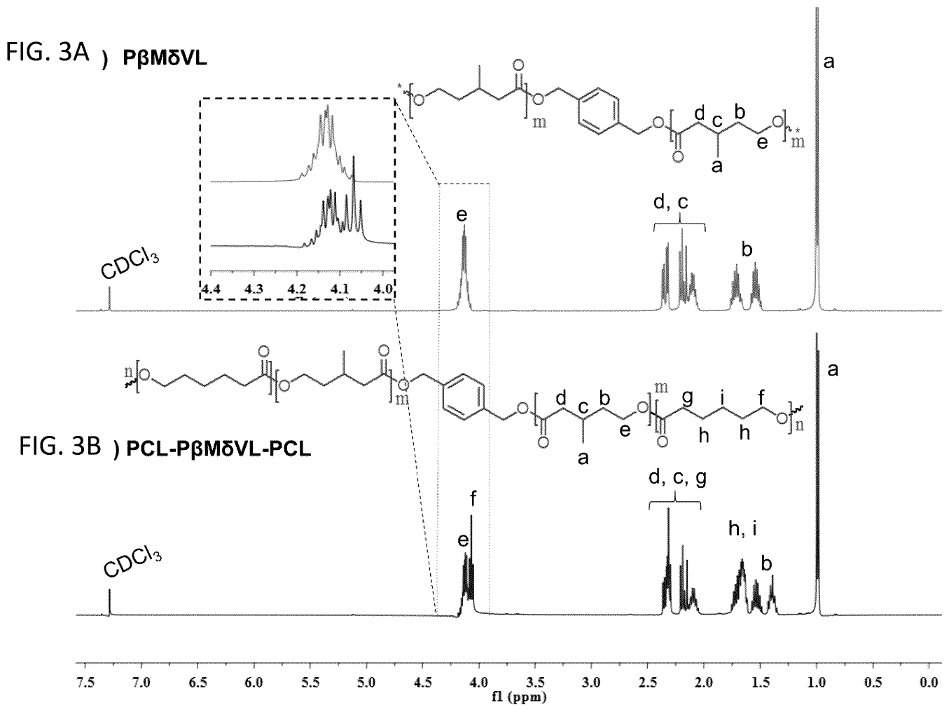

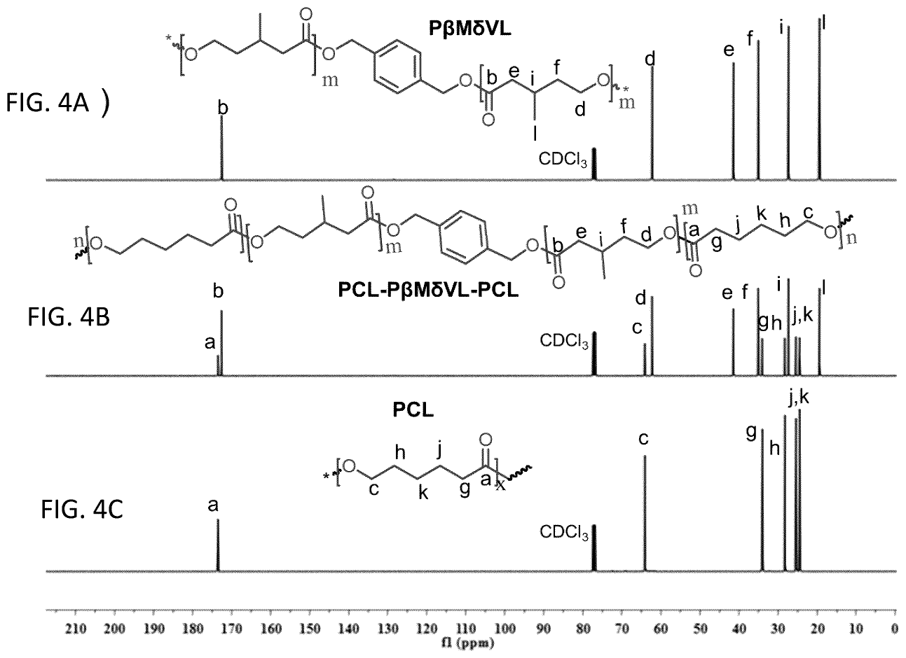

Successful synthesis of poly(β-methyl-δ-valerolactone) (PβMδVL) via ring-opening polymerization (ROP) of MVL was first validated through ¹H (FIG. 3A) and ¹³C NMR (FIG. 4A) analyses. The product displayed ¹H NMR signals for backbone methylene protons: at 2.32 ppm for (—CH₂CO, d), at 1.71 and 1.53 ppm for (—CH₂—; b), and at 4.13 ppm for (—CH₂O—; e) (FIG. 3A). The product also displayed ¹H NMR signals at 2.19 ppm for the backbone methane proton (—CH(CH₃); c) and at 0.99 ppm for the side-chain methyl protons (—CH₃; a). Furthermore, the appearance of a single resonance signal at 4.13 ppm instead of two anomeric signals at 4.23 and 4.08 ppm confirms successful lactone ring-opening. In addition, appearance of ¹³C NMR signals at 172.55 (—COO—, b), 62.21 (—CH₂O—; d), 41.42 (—CH₂CO—; e), 35.04 (—CH₂—; f), 27.35 (—CH(CH₃)—; i) and 19.47 (—CH₃; 1) also supported successful synthesis of PβMδVL (FIG. 4A).

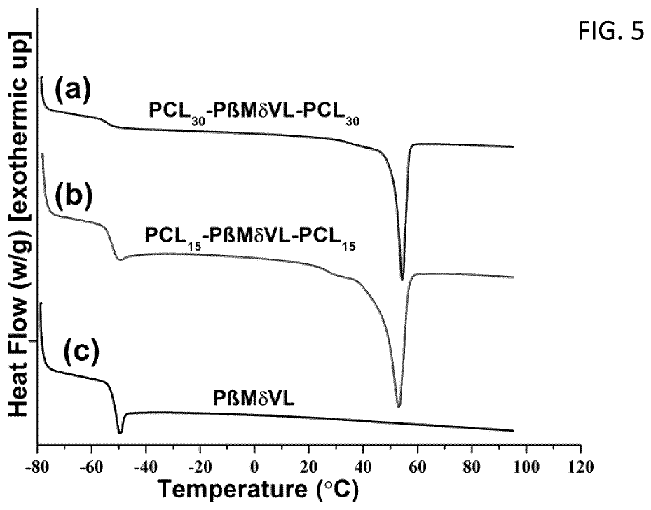

The GPC-MALS characterization of the synthesized PβMδVL revealed a number average molecular weight (Mn) of 62.72 kDa, close to the targeted molecular weight of 70 kDa (degree of polymerization DP=614), and a narrow dispersity of 1.01 (Table 1). The DSC analysis revealed a glass transition temperature (T_{g}) of −51.57° C. and absence of any meting peak (FIG. 5C). The synthesized PβMδVL is a telechelic diol, serving as a macroinitiator in triblock synthesis, and its low T_{g} and amorphous structure are required features to enable triblock polymers to have the elastomeric property at physiological temperature (37° C.).

TABLE 1
Compositional and physicochemical properties of the synthesized polymers.

| | | | | | Melting | ⁴Crystallinity |
|---|---|---|---|---|---|---|
| | ¹Mn | | | ³Tₘ | Enthalpy | of PCL |
| Polymers | (kDa) | ¹Ð | ²F_{CL} | (° C.) | (J/g) | endblocks (%) |
| βMδVL | 62.72 | 1.01 | 0 | N/A | N/A | N/A |
| PCL₁₀- | 82.31 | 1.13 | 0.24 | 52.2 | 13.99 | 41.78 |
| PβMδVL- | | | | | | |
| PCL₁₀ | | | | | | |
| PCL₁₅- | 90.64 | 1.12 | 0.30 | 52.9 | 18.92 | 45.20 |
| PβMδVL- | | | | | | |
| PCL₁₅ | | | | | | |
| PCL₂₀- | 94.92 | 1.14 | 0.34 | 53.4 | 21.88 | 46.13 |
| PβMδVL- | | | | | | |
| PCL₂₀ | | | | | | |
| PCL₃₀- | 100.03 | 1.23 | 0.37 | 54.3 | 28.64 | 55.48 |
| PβMδVL- | | | | | | |
| PCL₃₀ | | | | | | |

¹Mₙ = Number average molecular weight and Ð = dispersity, both determined by GPC;
²F_{CL} = fraction of PCL, calculated by comparing Mₙ of the polymer with that of βMδVL (e.g., F_{CL} for PCL₁₀-PβMδVL-PCL₁₀ = [(82.31 − 62.72)/82.31] = 0.24;
³Tₘ = melting temperature, evaluated from the differential scanning calorimetry (DSC).
⁴Calculated by comparing the melting enthalpy of the polymer with that of 100% crystalline PCL (139.5 J/g).

Synthesis of poly(caprolactone)-PβMδVL-poly(caprolactone) (PCL-PβMδVL-PCL) triblock polymers

Poly(caprolactone)-PβMδVL-poly(caprolactone) (PCL-PμMδVL-PCL) triblock polymers were synthesized via ROP of the commercially available caprolactone monomer with the telechelic PβMδVL diol as a macroinitiator, and the products were expected to contain two semi-crystalline PCL endblocks and an amorphous PβMδVL midblock (Scheme 1). Four compositionally different triblock polymers were synthesized by tuning the molecular weight of the PCL endblocks while keeping the same molecular weight for the PβMδVL midblock (62.72 kDa): PCL₁₀-PβMδVL-PCL₁₀, PCL₁₅-PβMδVL-PCL₁₅, PCL₂₀-PβMδVL-PCL₂₀, and PCL₃₀-PβMδVL-PCL₃₀, with the subscripted number adjacent to the PCL block representing its targeted molecular weight in kDa as seen in (Table 1).

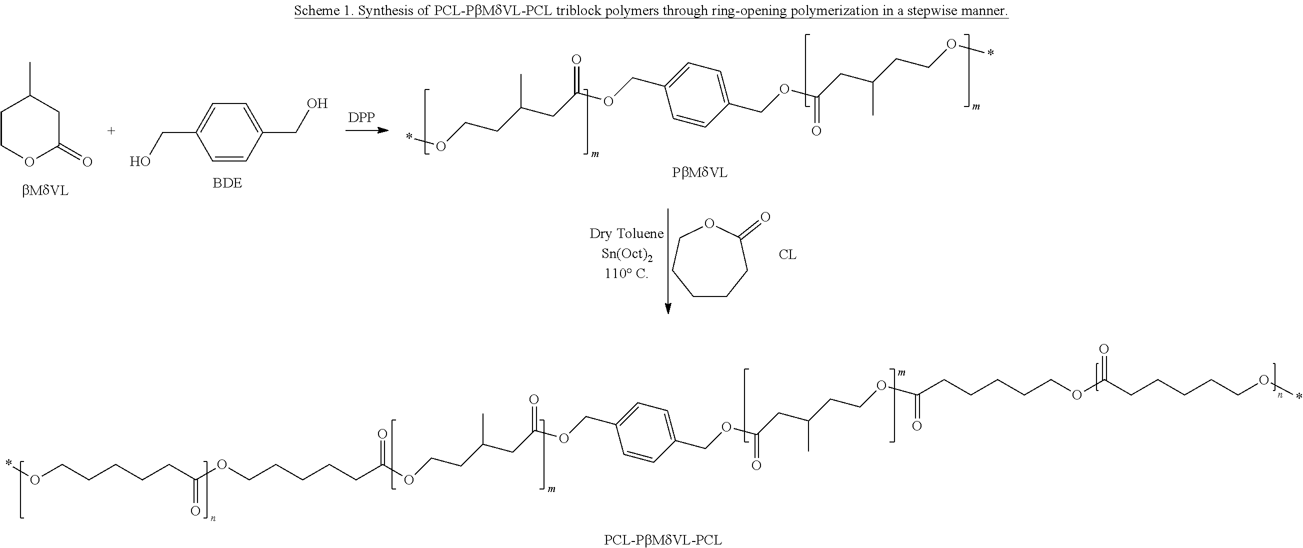

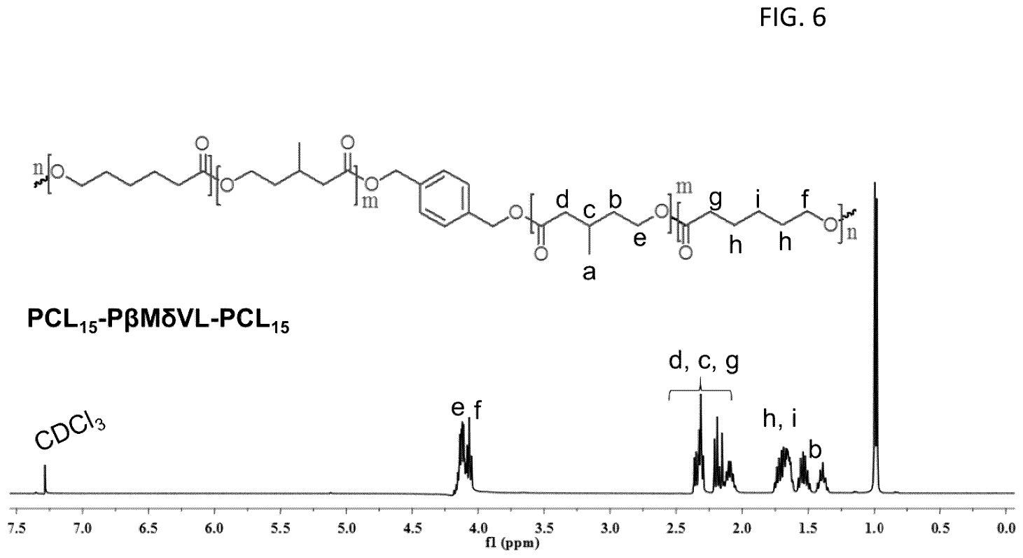

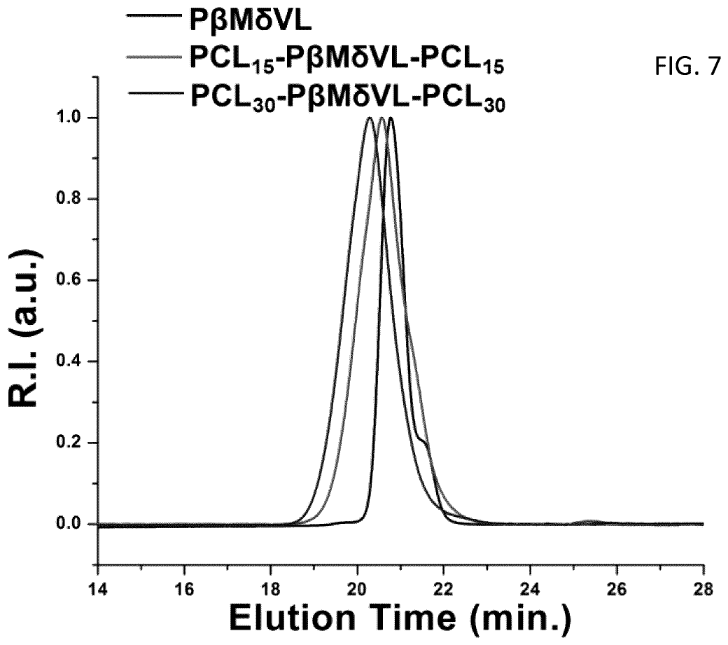

Successful synthesis of triblock polymers was suggested by NMR analyses. The polymers displayed ¹H NMR resonance signals at 4.13, 2.32, 2.19, and 0.99 ppm for the protons in the PβMδVL midblock as shown in FIG. 3B (and FIG. 6). The two adjacent resonance signals corresponding to the PβMδVL backbone methylene protons (—CH₂—; b) displayed a down field shift from 1.71 and 1.53 ppm to 1.57 and 1.39 ppm, respectively, indicating changes in the surrounding structural environment after formation of a triblock polymer. The signal corresponding to the methylene protons adjacent to the carbonyl group (—CH₂CO, g) in the PCL block appeared at 2.32 ppm, overlapping with the similar signal from CH₂CO in PβMδVL. The signals corresponding to the PCL backbone methylene protons, (—CH₂O—; f) and (—CH₂CH₂CH₂—; h and i), appeared at 4.05 and 1.66 ppm, respectively. The triblock polymers displayed ¹³C NMR signals at 172.55 (—COO—, b), 62.21 (—CH₂O—; d), 41.42 (—CH₂CO—; e), 35.04 (—CH₂—; f), 27.35 (—CH(CH₃)—; i) and 19.47 (—CH₃; 1) for the backbone carbons of the PβMδVL block (FIG. 4B). The ¹³C NMR signals for the carbons in the PCL backbone appeared at 173.48, 64.10, 34.08, 28.31, 25.46 and 24.54 ppm for (—COO—, a), (—CH₂O—; c), (—CH₂CO—g), (—CH₂—; h), (—CH₂—; j), and (—CH₂—; k), respectively, consistent with the signals displayed by the PCL homopolymer (FIG. 7C).

The GPC-MALS characterization of the synthesized triblock polymers revealed a shift toward shorter retention times as compared with PβMδVL, suggesting increased molecular weights and successful synthesis of the triblock polymers (FIG. 7). Furthermore, the retention time of PCL₃₀-PβMδVL-PCL₃₀ shifts to a lower value as compared with that of PCL₁₅-PβMδVL-PCL₁₅, suggesting that compositionally different triblock polymers were synthesized as planned (FIG. 7). The results of NMR analyses and GPC-MALS characterization together confirmed that the designed PCL-PβMδVL-PCL triblock polymers were successfully synthesized.

### Thermal Properties of the Block Polymers

The DSC characterization for triblock polymers revealed both glass transition and melting peaks (for the amorphous and crystalline components, respectively), in contrast to the absence of any melting peak for amorphous PβMδVL, suggesting that the PCL endblocks were crystalline or semi-crystalline (FIG. 5). The crystallinity (X_{c}) of the PCL block in each triblock polymer was determined by calculating the ratio of its melting enthalpy to 139.5 J/g, which is the melting enthalpy of fully crystalline PCL. The crystallinity was 41.78%, 45.20%, 46.13%, and 55.48% for PCL₁₀-PβMδVL-PCL₁₀, PCL₁₅-PβMδVL-PCL₁₅, PCL₂₀-PβMδVL-PCL₂₀ and PCL₃₀-PβMδVL-PCL₃₀, respectively (Table 1). The crystallinity increases with the endblock length, because the crystalline structure of the PCL segment adjacent to the amorphous PβMδVL midblock is disrupted and the percentage of the disrupted segment decreases with the increasing PCL block length. The crystalline structure in the PCL endblocks is essential for the triblock polymers to exhibit mechanical strengths, as these crystalline domains serve as junctions to convey mechanical properties.

The DSC curves revealed a glass transition temperature (T_{g}) of −51.75° C. for amorphous P3MSVL. This low T_{g} allows the midblock chains to be in the rubbery state at the application temperature of 37° C., enabling the triblock polymers to display the elastomeric property. The melting temperature (Tₘ) of the triblock polymers increases with the increasing PCL block length (52.25° C. and 54.36° C. for PCL₁₀-PβMδVL-PCL₁₀ and PCL₃₀-PβMδVL-PCL₃₀, respectively), probably due to increased crystallinity (Table 1)(Haitao Qian, J. B. a. S. W. Synthesis, characterization and degradation of ABA block copolymer of L-lactide and F-caprolactone. Polymer Degradation and Stability 2000, 68, 423-429, DOI: https://doi.org/10.1016/S0141-3910(00)00031-8). All the triblock polymers reported in this study have a Tm below 55° C. (Table 1), suggesting that a processing temperature of 55° C. would be sufficient to process these thermoplastic polymers to desire shapes.

### Mechanical Properties of the Block Polymers

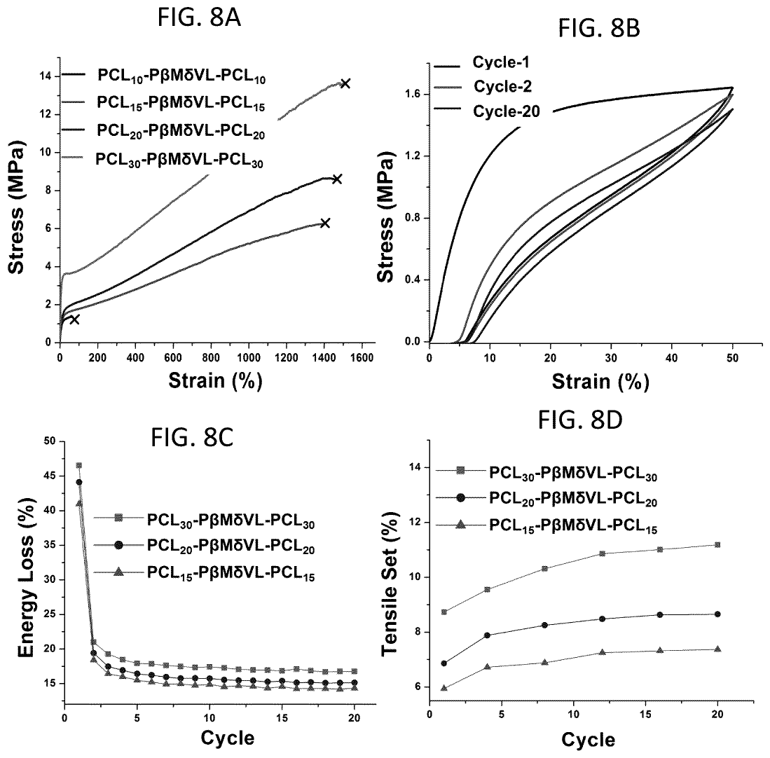

The uniaxial stress-strain curves of the triblock polymers are shown in FIG. 8A. The Young's modulus calculated from the lower strain region (0-5%) of each stress-strain curve ranges from 12 to 48 MPa as summarized in Table 2, and it increases with the PCL block length, probably due to the increased crystallinity (Nasiri, M.; Saxon, D. J.; Reineke, T. M. Enhanced Mechanical and Adhesion Properties in Sustainable Triblock Copolymers via Non-covalent Interactions. Macromolecules 2018, 5] (7), 2456-2465, DOI: 10.1021/acs.macromol.7b02248). The ultimate tensile strength also increases with the PCL block length from 1.3 MPa for PCL₁₀-PβMδVL-PCL₁₀ to 13.6 MPa for PCL₃₀-PβMδVL-PCL₃₀. The strain to failure is greater than 1000% for PCL₁₅-PβMδVL-PCL₁₅, PCL₂₀-PβMδVL-PCL₂₀, and PCL₃₀-PβMδVL-PCL₃₀, suggesting that these polymers are highly stretchable (Table 2). The strain to failure for PCL₁₀-PβMδVL-PCL₁₀ is only 66%, and its ultimate tensile strength is much lower than those of the other three polymers, suggesting that the junctions formed from 10 kDa PCL endblocks are not strong enough to allow extensive stretching.

TABLE 2
Mechanical properties of the synthesized polymers

| | | ²Tensile | Ultimate | Strain to |
|---|---|---|---|---|
| | ¹E | Set | strength | failure |
| Polymers | (MPa) | (%) | (MPa) | (%) |
| βMδVL | N/A | N/A | N/A | N/A |
| PCL₁₀-PβMδVL- | 12.78 ± | N/A* | 1.39 ± | 66 ± |
| PCL₁₀ | 2.05 | | 0.4 | 9.12 |
| PCL₁₅-PβMδVL- | 14.76 ± | 7.37 ± | 6.24 ± | 1394 ± |
| PCL₁₅ | 1.39 | 0.42 | 0.68 | 20.98 |
| PCL₂₀-PβMδVL- | 16.78 ± | 8.65 ± | 8.59 ± | 1458 ± |
| PCL₂₀ | 0.91 | 0.49 | 0.51 | 28.37 |
| PCL₃₀-PβMδVL- | 48.52 ± | 11.18 ± | 13.60 ± | 1502 ± |
| PCL₃₀ | 7.26 | 0.78 | 2.17 | 41.25 |

¹E = Young's modulus;
²Tensile set (defined as the residual strain when unloading reaches 0 stress) at the 20th cycle.
*PCL₁₀-PβMδVL-PCL₁₀ was broken during the 2^{nd} cycle.

The stress-strain curves during 20 cycles of cyclic tensile loading (50% strain) and unloading revealed that the hysteresis energy loss during the first cycle was relatively large (42-47%), but became significantly smaller for all subsequent cycles (14-17% at cycle 20) (FIG. 8B, 8C). Rubber bands exhibit similar behavior: the hysteresis energy loss is approximately 22% during the first cycle, and it drops to one third of that after cycle 5.¹² Among the 3 stretchable triblock polymers, PCL₁₅-PβMδVL-PCL₁₅ exhibited the least hysteresis energy loss. The PCL₁₅-PβMδVL-PCL₁₅ polymer also showed the smallest tensile set (7.37% at the 20^{th} cycle), which is defined as the residual strain when the unloading cycle reaches 0 stress (FIG. 8D). The increase in the tensile set was most significant in the first cycle, and only a minor increase was observed in each of subsequent cycles. This is also consistent with the behavior of other elastomeric materials such as rubber bands.¹² The PCL₁₅-PβMδVL-PCL₁₅ material exhibited lower hysteresis energy loss and tensile set than PCL₂₀-PβMδVL-PCL₂₀ and PCL₃₀-PβMδVL-PCL₃₀, suggesting that the length of the semi-crystalline PCL endblocks needs to be optimized: as long as they are long enough to form strong junctions to allow extensive stretching, shorter endblocks endow better elastomeric properties.

### Cytotoxicity of the Elastomeric Polymers

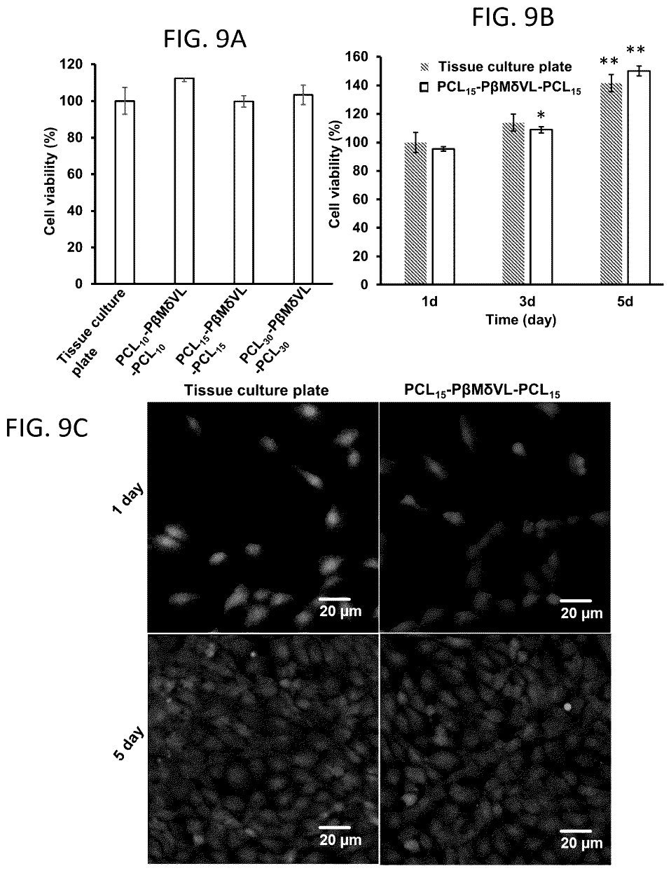

Cytotoxicity of the polymers was evaluated using the Alamar Blue assay and live-dead cell staining. The Alamar Blue assay revealed that the viability and proliferation of the cells cultured on the elastomeric triblock polymers are comparable to those on the tissue culture polystyrene control (FIG. 9A, 9B). Live-dead cell staining showed that the morphology of the cells cultured on these polymers is similar to that on tissue culture polystyrene, and few dead cells were observed in all the samples and controls (FIG. 9C). These results suggest that the elastomeric polymers reported in this study have no cytotoxicity.

### 3D Printing

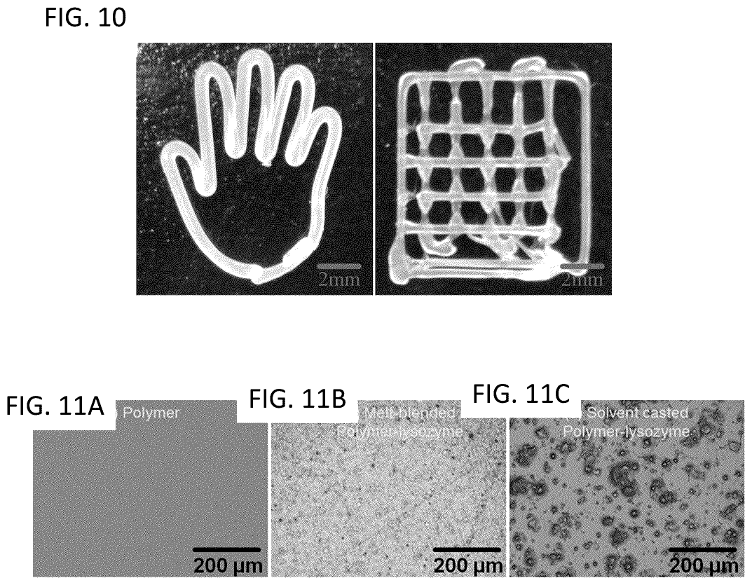

The thermoplastic nature of the elastomers reported in this study and their melting temperatures around 55° C. make it easy to process these polymers into desired shapes and structures. We demonstrated that these elastomers could be easily 3D printed by commonly used fused deposition modeling (FDM) technology on a BIO-X 3D printer equipped with a thermoplastic head (Guvendiren, M.; Molde, J.; Soares, R. M.; Kohn, J. Designing Biomaterials for 3D Printing. ACS biomaterials science & engineering 2016, 2 (10), 1679-1693, DOI: 10.1021/acsbiomaterials.6b00121.) The PCL₁₅-PβMδVL-PCL₁₅ polymer was fed into the printer head and heated to 60° C., and the polymer melt was printed with a pressure of 200 kPa to form a hand-shaped structure and a square-shaped structure (FIG. 10). It is expected that the precision of the printed structures can be improved by tuning the nozzle gauge and printing speed.

### Protein Encapsulation in the Elastomeric Polymers

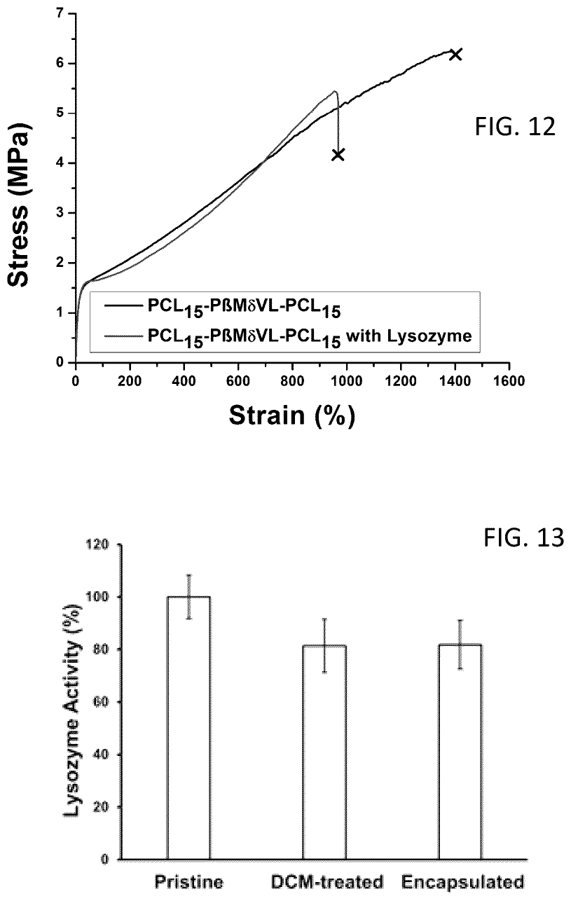

Due to the thermoplastic nature and a melting temperature much lower than 100° C., these elastomers are expected to be particularly attractive for fabrication of devices or tissue engineering scaffolds that encapsulate bioactive molecules. Many bioactive molecules, such as proteins, lose their bioactivities when exposed to high temperatures, and therefore materials that allow encapsulation of these bioactive molecules through a simple processing procedure involving a relatively mild temperature are highly desirable. Lysozyme, which has a denaturation temperature of 75° C., (Goyal, M. K.; Roy, I.; Amin, A.; Banerjee, U. C.; Bansal, A. K. Stabilization of lysozyme by benzyl alcohol: surface tension and thermodynamic parameters. Journal of pharmaceutical sciences 2010, 99 (10), 4149-61, DOI: 10.1002/jps.22129) was chosen as a model agent to be encapsulated in the PCL₁₅-PβMδVL-PCL₁₅ polymer via melt-blending at 60° C. in a twin-screw extruder. The melt-blended product was further hot-pressed to 0.2 mm thick films at 60° C. and 350 kPa. Optical microscopy revealed that lysozyme particles were distributed uniformly throughout each specimen (FIG. 11B) In contrast, the distribution of lysozyme particles encapsulated in PCL₁₅-PβMδVL-PCL₁₅ films through solvent casting was not uniform (FIG. 11C). Uniaxial tensile tests showed that the Young's modulus of the PCL₁₅-PβMδVL-PCL₁₅ polymer containing 18.25% lysozyme was 14.68 MPa (FIG. 12, Table 3), similar to that of the pristine polymer (14.76 MPa). The strain to failure was 959% and the ultimate tensile strength was 5.46 MPa, both dropping slightly as compared with the values for the pristine polymer (FIG. 11A).

TABLE 3
Mechanical properties of pristine PCL₁₅-PβMδVL-PCL₁₅ and
PCL₁₅-PβMδVL-PCL₁₅ encapsulating lysozyme.

| | Ultimate | Strain to | |
|---|---|---|---|
| | strength | failure | E |
| Polymers | (MPa) | (%) | (MPa) |
| PCL₁₅-PβMδVL-PCL₁₅ | 6.24 ± | 1394 ± | 14.76 ± |
| | 0.68 | 20.98 | 1.39 |
| PCL₁₅-PβMδVL-PCL₁₅ | 5.46 ± | 959 ± | 14.68 ± |
| with lysozyme (18.25 wt %) | 0.38 | 6.42 | 1.01 |

The activity of the lysozyme encapsulated in the film was examined to assess the effect of the double processing procedures at 60° C. (melt-blending and hot-pressing). The film was treated with DCM to dissolve the polymer, followed by extraction of lysozyme with water. Two controls, one with pristine lysozyme and one with DCM-treated lysozyme, were conducted. The activity of the lysozyme extracted from the polymer was approximately 18% lower than that of pristine lysozyme, but almost the same as that of DCM-treated lysozyme (FIG. 13). This result suggests that lysozyme lost its activity when exposed to DCM, but the double processing procedures at 60° C. did not affect its activity. Therefore, the thermoplastic elastomers reported in this study will allow many bioactive molecules, as long as they do not lose activities at 60° C., to be readily encapsulated with preserved bioactivities.

### Degradation of the Elastomers

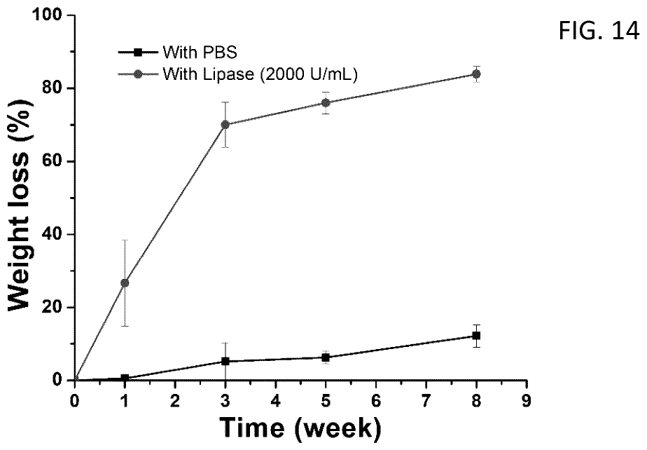

The elastomers reported in this study have a hydrolysable polyester backbone. Hydrolysis of PCL₃₀-PβMδVL-PCL₃₀ in PBS and in a lipase solution (Thermomyces lanuginosus; 2000 U/mL) was examined. Disk-shaped specimens (0.2 mm thick, 8 mm in diameter) were incubated in each solution at 37° C. for up to 8 weeks. In PBS, the weight loss was almost negligible at 1 week and approximately 11% at 8 weeks, and it increased almost linearly between 1 and 8 weeks, suggesting a combination of bulk degradation and surface erosion (FIG. 14). In the lipase solution, polymer degradation was significantly accelerated, as hydrolysis of the ester bond can be accelerated by lipase (FIG. 14) (Suming Li, L. L., Henri Garreau, and Michel Vert. Lipase-Catalyzed Biodegradation of Poly(E-caprolactone) Blended with Various Polylactide-Based Polymers. Biomacromolecules 2003, 4, 372-377, DOI: 10.1021/bm025748j).

What is claimed is:

1. A method of using a block copolymer, the method comprising:
providing a block copolymer, the block copolymer comprising at least a polycaprolactone (PCL) block; and
a second block being amorphous and having a glass transition temperature less than 30° C.; and
subjecting the block copolymer to thermal processing at a temperature less than 100° C.

2. A method according to claim 1, wherein the second block comprises a poly(P-methyl-6-valerolactone) (PMVL) block.

3. A method according to claim 1, wherein the second block comprises amorphous poly(caprolactone-co-lactide), poly(lactide-co-trimethylene carbonate), polyurethane, poly(trimethylene carbonate), poly(ester ether), polyhydroxyalkanoates, or combinations thereof.

4. A method according to claim 2, wherein the PMVL block has a mass average molar mass (M_{w}) of 10 kDa or greater.

5. A method according to any of claims 1 to 4, wherein the thermal processing occurs at a temperature less than 70° C.

6. A method according to any of claims 1 to 4, wherein the thermal processing occurs at a temperature less than 65° C.

7. A method according to any of claims 1 to 4, wherein the thermal processing occurs at a temperature from 50° C. to 60° C.

8. A method according to any of the claims 1 to 4 further comprising combining a bioactive agent with the block copolymer before, while being subjected to thermal processing, or both.

9. A method according to claim 8, wherein the thermal processing is three dimensional (3D) printing, extrusion, molding, or some combination thereof.

10. A method according to claim 8, wherein the bioactive agent is a protein.

11. A method according to claim 8, wherein each block has an M_{w} of about 10 kDa or greater.

12. A method according to claim 8, wherein the PMVL block has a ¹⁴C/¹²C ratio greater than zero.

13. A method according to claim 8, wherein the block copolymer has a ¹⁴C/¹²C ratio greater than zero.

14. A method according to claim 8, wherein the block copolymer is an ABA block copolymer, wherein the mid-block comprises a second block being amorphous and has a glass transition temperature less than 30° C., and wherein the end blocks are the PCL blocks.

15. The method according to claim 14, wherein the mid-block comprises amorphous poly(caprolactone-co-lactide), polyurethane, poly(trimethylene carbonate), Poly(ester ether), and random copolymers formed from caprolactone and any other monomers that can disrupt the crystalline structure of the resulting polymers

16. An article comprising:
a block copolymer, the block copolymer comprising at least a polycaprolactone (PCL) block, and a second block having a glass transition temperature less than 30° C.; and
at least one bioactive agent,
wherein the article was formed using a thermal processing method at a temperature of less than 100° C.

17. The article according to claim 16, wherein the at least one bioactive agent is selected from: growth factors, cytokines, small molecules and combinations thereof.

18. The article according to any of claims 16 to 17, wherein the growth factors, cytokines, or combinations thereof are selected from: vascular endothelial growth factor (VEGF), Platelet-derived growth factor (PDGF), basic fibroblast growth factor (bFGF), acidic fibroblast growth factor (aFGF), hepatocyte growth factor (HGF), insulin-like growth factor-1 (IGF-1), stromal-cell derived factor 1 (SDF-1), nerve growth factor (NGF), neurotrophin 3 (NT-3), brain-derived neurotrophic factor (BDNF), glial-derived neurotrophic factor (GDNF), glial growth factor (GGF), ciliary neurotrophic factor (CNTF), leukemia inhibitory factor (LIF), growth differentiation factor 5 (GDF-5), Erythropoietin (EPO), interleukin-4 (IL-4), interleukin-2 (IL-2), interferon gamma (IFN-γ), and combinations thereof.

19. The article according to claim 16 to 17, wherein the bioactive agent is a small molecule.

20. The article according to claim 17, wherein the small molecule is selected from the group consisting of: paclitaxel, carmustine, gemcitabine, histrelin, leuprolide, goserelin, corticosteroids, simvastatin, risperidone, tacrolimus, vancomycin, lidocaine, buprenorphine, hydromorphine, levonorgestrel, estradiol, etonogestrel, and combinations thereof.

21. The article according to any of claims 16 to 17, wherein the article has an elastic modulus from 0.01 MPa to about 2500 MPa.

22. The article according to any of claims 16 to 17, wherein the article has an elastic modulus from about 1.5 MPa to about 300 MPa.

23. The article according to any of claims 16 to 17, wherein the article has a percent elongation of from about 5% to about 5000%.

24. The article according to any of claims 16 to 17, wherein the article has a percent elongation from about 300% to about 2000%.

25. The article according to any of claims 16 to 17, wherein the article is formed using 3D printing, extrusion, molding, or some combination thereof
